(19) United States
(12) Patent Application Publication
(10) Pub. No.: US 2017/0065731 A1
(43) Pub. Date: Mar. 9, 2017

(54) Method, Apparatus, and System for Radiation Therapy

(71) Applicant: Medical Theranostics Inc., Monarch Beach, CA (US)

(72) Inventors: Shyam M. Srinivas, Monarch Beach, CA (US); Aniruddha K. Doke, Highland Heights, OH (US)

(21) Appl. No.: 15/179,997

(22) Filed: Jun. 11, 2016

Related U.S. Application Data
(63) Continuation of application No. 14/846,817, filed on Sep. 6, 2015, now abandoned.

(51) Int. Cl. A61K 51/12; G01T 1/02

(52) U.S. Cl. CPC A61K 51/1251; G01T 1/02

(57) ABSTRACT

A device and method for radioembolization in the treatment of cancer cells in the body. In preferred embodiments, the device comprises at least two isotopes; wherein a first isotope is focused on therapeutic purposes and a second isotope is focused on dosimetric imaging purposes. In further embodiments, the first isotope is a radiation emitter for therapy where the radiation emitted is primarily alpha particles, and the second isotope is a positron emitter for PET imaging. In preferred embodiments, the isotopes are bound to a single resin microsphere, and an after a radiation dose using the present invention, both treatment and treatment efficacy can be provided to a cancer patient.

### FIELD OF THE INVENTION

This invention relates to the imaging and treatment of cancer using radioactive polymeric particles. In particular aspect, the invention relates to use of microspheres having radionuclides.

### BACKGROUND OF THE INVENTION

Hepatocellular carcinoma (“HCC”) is the third leading cause of cancer related deaths and the sixth most prevalent cancer as ˜750,000 new cases are diagnosed each year that result in ˜700,000 deaths worldwide. Axelrod and von Leeuwen have reported that the incidence of HCC has “more than doubled, from 2.6 to 5.2 per 100,000 population” over the past 20 years, with an increase in mortality from 2.8 to 4.7 per 100,000. 80% of the HCC cases are due to the early acquisition of hepatitis B and C in conjunction with high-risk behavior. Additionally, the obesity epidemic has contributed to an increase in non-alcoholic steatohepatitis (NASH), which can eventually progress to fibrosis, cirrhosis, and HCC.

The treatment of HCC has been challenging, since most patients present at an advanced stage. Symptoms of liver cancer are often vague and don't appear until the cancer is at an advanced stage. In early stages of the disease, surgical treatments like resection and transplantation provide the best curative outcomes. A disadvantage of resection, however, is that patients' remnant livers may not be able to support the necessary hepatic functional demands, and there is a high potential for recurrent disease. Moreover, there are ˜35,000 patients diagnosed with HCC annually in the US alone of which ˜80% have disseminated unresectable tumors. Other treatment modalities include transarterial chemoembolization (TACE), sorafenib chemotherapy, external beam radiation, and radiofrequency ablation. In comparison to sorafenib and external beam radiation, more local therapies such as radiofrequency ablation, radioembolization (RE), and TACE are able to deliver the desired dose to the target with minimal toxicity to the system. Supporting this statement, Dezarn et al. noted that the maximum external beam acceptable dose to the whole liver of 35 Gy delivered in 1.8 Gy/day fractions is far below the 70 Gy typically needed to destroy solid tumor lesions. The high sensitivity of normal hepatic tissue to external beam radiation has given way to more locally effective RE or selective internal radiation therapy (SIRT) techniques.

RE, a promising catheter based liver-directed modality indicated for HCC, is the transcatheter angiographic delivery of microspheres. Injection of the microspheres via the hepatic artery provides advantage as observations have demonstrated that metastatic hepatic malignancies >3 mm derive ˜80-100% of their blood supply from the arterial rather than the portal hepatic circulation. The normal liver tissue is predominantly fed by the portal vein (60-70%). Current Yttrium-90 (⁹⁰Y) spheres trapped at the precapillary level emit internal β radiation, providing a relatively more localized higher dose delivery as compared to external beam radiation. However, the average energy of 0.94 MeV of ⁹⁰Y β emission that delivers ˜49.38 Gy/kg/GBq to tissue, and its longer path length (mean tissue penetration of 2.5 mm and a maximum range of 1.1 cm) result in collateral damage of the normal liver. Radiation damage due to the longer path length is amplified as ˜25% of liver is also fed by the hepatic artery. Thus, normal liver tissue can receive doses that are non-trivial with ensuing side effects. Side effects from β radiation to the normal liver can cause nausea, discomfort and liver dysfunction. Abnormal high radiation doses to the normal tissue may even result in radiation-induced hepatitis with potential risk of liver failure.

The other disadvantage of prior art ⁹⁰Y microspheres is their inability to be imaged easily with high quality and quickly acquired images on par with other currently used diagnostic isotopes. Dosimetry in ⁹⁰Y radioembolization is largely empirical when following current manufacturer recommended guidelines of both glass and resin microsphere products. Little is known about the actual radiation absorbed dose to both tumor and normal liver in these treatments. Thus, the prior art dosing regimen is suboptimal for tumor kill, sparing of normal liver, and does not take advantage of the newer techniques of quantitative PET/CT imaging for accurate dosimetry.

### SUMMARY OF THE DISCLOSURE

The present invention discloses a device and method for radioembolization in the treatment of cancer cells in the body. In a specific embodiment, the targeted organ is the liver and the disease state that is being treated is hepatocellular carcinoma (HCC). In preferred embodiments, the device comprises at least two isotopes; wherein a first isotope is focused on therapeutic purposes and a second isotope is focused on dosimetric imaging purposes. In further embodiments, the first isotope is a radiation emitter for therapy where the radiation emitted is primarily alpha particles, and the second isotope is a positron emitter for PET imaging. In further embodiments, the first isotope is Actinium-225 (²²⁵Ac), and the second isotope is Zirconium-89 (⁸⁹Zr). In accordance with the preferred embodiments of the present invention, the method and device has at least 5 times greater tumoricidal efficacy than existing 90Y radioembolization techniques. In further embodiments of the present invention, an amount of radiation dose absorbed to both tumor cells and normal liver cells after radioembolization can be determined from the device within 5 minutes of the start of the PET imaging. In preferred embodiments, the isotopes are bound to a single resin microsphere, and contain a total number of particles around 37 million in each radiation dose.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIG. 1 is a schematic view of the radiomicrosphere used for RE in accordance with a preferred embodiment of the present invention; and

FIG. 2 is an illustrative cross sectional view of a liver sinusoid with use of the radiomicrosphere in FIG. 1 in accordance with a preferred embodiment of the present invention.

### DETAILED DESCRIPTION OF THE PREFERRED EMBODIMENTS

As shown in the drawings for purposes of illustration, the invention is embodied in a new radiomicrosphere for use with radioembolization. In preferred embodiments of the present invention, the new radiomicrosphere can significantly extend the overall survival for patients with primary liver cancer also known as hepatocellular carcinoma (HCC), while providing distinct advantages and features over the prior art. However, it will be recognized that further embodiments of the invention may be used for other disease states including cancer in other parts of the body.

The current prior art when treating HCC using radioembolization (RE) is the transcatheter angiographic delivery of Yttrium-90 (⁹⁰Y) microspheres. The disadvantages with ⁹⁰Y RE are the high beta radiation to the normal liver (collateral damage), suboptimal dosing regimens for tumor kill and the lack of quantitative imaging for accurate dosimetry. The preferred embodiments of the present invention replaces ⁹⁰Y microspheres with a new theranostic (i.e. therapeutic+diagnostic) agent designed to be the next generation of radiomicrosphere.

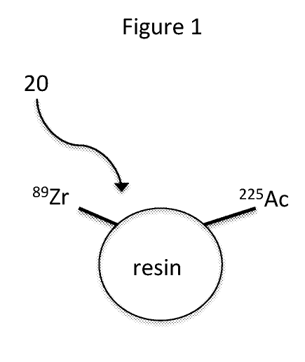

FIG. 1 illustrates the radiomicrosphere 20 in accordance with the preferred embodiments of the present invention. Radiomicrosphere 20 is the first theranostic sphere in commonly used RE products. In preferred embodiments, radiomicrosphere 20 is a resin microsphere with both a diagnostic isotope, Zirconium-89 (⁸⁹Zr), a positron emitter for PET imaging, and a therapeutic isotope, Actinium-225 (²²⁵Ac), an alpha emitter for therapy. The innovation lies in the dual nature of the agent that offers both focal therapy and quantitative dosimetry simultaneously for the treatment of HCC. The advantage of radiomicrospheres 20 over existing ⁹⁰Y radiomicrospheres is the greater tumoricidal effect with a lesser amount of radiation (and thus less collateral damage) as well as provide valuable dosimetry and diagnostic information. On the therapeutic aspect, radiomicrospheres 20 is advantageous over existing ⁹⁰Y radiomicrospheres as the ²²⁵Ac alpha particle will have greater tumoricidal efficacy with a lesser amount of radiation due to the 5×-10× greater biological effect from alpha particles vs. ⁹⁰Y beta particles. The lethal dose is delivered by the four alpha particles in the decay chain of ²²⁵Ac. Alpha particles result in a greater number of double stranded DNA breaks, which are lethal events for cells as opposed to a single stranded DNA break from a beta particle that is more easily repairable. In addition, alpha particles result in less “collateral damage” due to their greater mass and shorter path length in tissue. The estimated path length of ²²⁵Ac alpha particle is 80-100 μm where a ⁹⁰Y beta particle is on the order of 1-2.5 mm. Also, the two betas from ²²⁵Ac decay have much lower energy (444 keV, 659 keV and 198 keV) and mean path lengths (first β 98% chance of 1.2 mm; 2% chance of 1.8 mm; second β 100% chance of 0.5 mm). A good example of fewer side effects from alpha emitter therapy when compared to beta emitters, is the current clinical use of ²²³Ra, the first FDA approved alpha emitter for the treatment of osseous metastases in prostate cancer. ²²³Ra has a much kinder side effect profile with less bone marrow suppression than its beta emitter predecessors namely ¹⁵³Sm and ⁸⁹Sr.

According to a preferred embodiment of the present invention, a uniqueness of this radiomicrosphere 20 construct is to have the diagnostic information obtained simultaneously once the therapy has been deployed. The quantitative power of PET can be utilized to determine the radiation dose absorbed to both tumor and normal liver. Traditionally in ⁹⁰Y RE, post therapy imaging is not part of the treatment paradigm in a clinical setting. Although positron emission does occur in ⁹⁰Y, its positron branching fraction is extremely small (32 ppm), making it a non-ideal imaging agent and also requires patients to lay still in a PET/CT scanner for 20-30 minutes for image acquisition. Contrast that with ⁸⁹Zr which has a 22.6% positron branching fraction, making it an ideal imaging agent. PET/CT images from ⁸⁹Zr can be collected in a 3-7 minute time span and their quality will be far superior because of the higher positron count rate. The concurrent PET imaging of the treatment would offer valuable information to understanding the efficacy, dosage of the treatment and also the damage to the normal liver. This quantitative information can guide the future management of the patient, alerting clinicians when follow up treatments or closer surveillance of liver function may be necessary. Furthermore, the quantitative power of PET imaging can show whether tumors respond to such radiation treatment. It has been discovered that even after receiving what would be considered tumoricidal doses of radiation, many tumors do not respond. The quantitative information from the ⁸⁹Zr PET/CT scan will come from converting the activity on the scan in Bq to the actual radiation absorbed dose in Gy within a defined volume of interest. A local deposition method using MIRD dosimetry or a convolution kernel can be employed both accepted methods for converting activity to radiation absorbed dose. This scan will give the macroscopic information about dose to tumors and to normal liver tissue.

In alternative embodiments of the present invention, alternative isotopes may be substituted without taking away from the spirit of the invention. Examples of alternative isotopes include:

Alpha emitters:

Terbium-149 (Tb-149)

Astatine-211 (At-211)

Bismuth-212 (Bi-212)

Bismuth-213 (Bi-213)

Radium-223 (Ra-223)

Radium-224 (Ra-224)

Thorium-227 (Th-227)

Thorium-228 (Th-228)

Fermium-255 (Fm-255)

Positron emitters:

Fluorine-18 (F-18)

Scandium-44 (Sc-44)

Copper-64 (Cu-64)

Gallium-68 (Ga-68)

Yttrium-86 (Y-86)

Iodine-124 (1-124)

Terbium-152 (Tb-152)

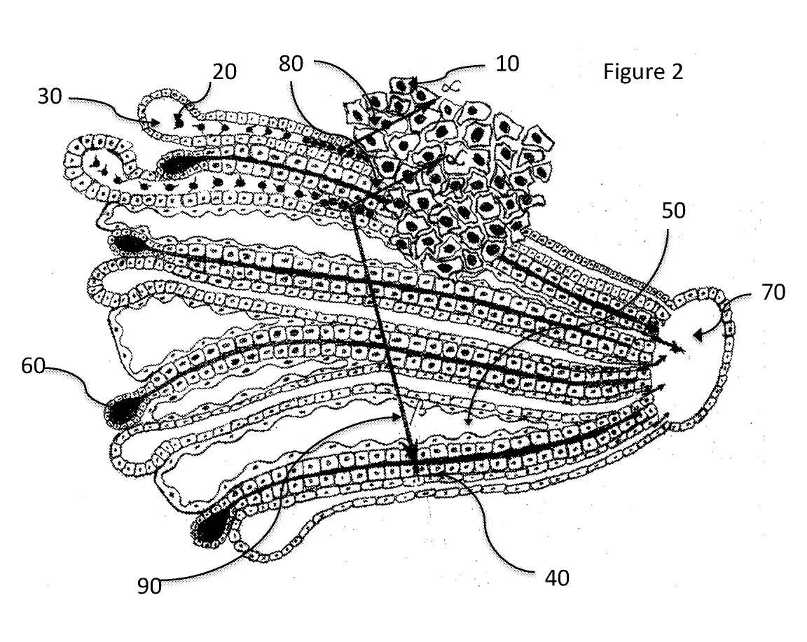

FIG. 2 is an illustrative cross sectional view (highly magnified, not to scale) of a liver sinusoid with use of the radiomicrosphere 20 in FIG. 1 in accordance with preferred embodiments of the present invention. As per RE techniques, the AlphaSphere radiomicrospheres 20 are injected intra-arterially into the hepatic arteriole 30 near the targeted area of the liver to treat the hepatocellular carcinoma (HCC) 10. A more detailed description of the RE procedure will be described below. For reference purposes, FIG. 2 is labeled with parts of the liver including normal hepatocytes 40, portal venule 50, bile canaliculi 60, and central vein 70. As seen in FIG. 2, the alpha particle pathway 80 is shown to illustrate the mean free path which is more localized and focused around the HCC tumor 10. For illustrative purposes, the beta particle pathway 90 is shown to illustrate that the relative longer distance the beta particle will travel in the liver.

The typical sequence of events for a liver cancer patient is as follows: The patient will usually exhibit symptoms or laboratory abnormalities suspicious for a liver tumor. The patient will have CT or MM imaging to document the presence of a lesion. That lesion will be biopsied to confirm a cancer. Depending on the stage of the cancer, surgery may not be an option (roughly 80% of the time). The patient will be referred to an interventional radiologist for radioembolization treatment. Further specialized imaging may be obtained (i.e. PET/CT, octreotide SPECT, triphasic liver CT or MM) to better characterize the extent of the tumor. The interventional radiologist will then conduct an anatomic mapping of the patient's liver vasculature. This is essentially an exploration of the vasculature within the liver and discovering which vessel(s) are the optimal route of approach for delivery of the radiomicrospheres 20.

Once the interventional radiologist has placed a catheter in the location which he believes is optimal for delivery, a simulation is performed by injecting technetium-99m tagged to macroaggregated albumin (Tc^{99m}-MAA) as a precursor to the intended RE therapy. The advantage of this agent is that the MAA has a similar size (˜30 micron diameter) when compared to the radiomicrospheres 20, and thus the MAA distribution is a good simulation for the eventual distribution of the injected radiomicrospheres 20. The MAA being tagged with Tc^{99m} is readily amenable to SPECT/CT imaging, taking advantage of the anatomical landmarks that come with hybrid imaging. The MAA (being a protein) is digested by enzymes, and therefore its embolization of the tumor vasculature is only transient enough for the imaging. Once images are acquired the MAA is degraded making way for the radiomicrospheres 20 that are to follow in another angiographic session. On occasion the MAA scan may reveal some shunting of blood to other normal organs from the liver vessels. This can manifest itself as uptake in the stomach, duodenum, or sometimes as excessive lung shunting. Such undesirable uptake usually can be prevented with coil embolization to cut off those vascular pathways prior to the RE treatment. In the case of excessive lung shunting, the radiomicrospheres 20 dose would be reduced.

When the patient returns on the actual therapy day, an authorized user of radioactivity (either a radiation oncologist or a nuclear medicine physician) will have calculated the appropriate dose for the patient and will administer this to the patient through the interventional radiologist's catheter after it is placed in the same position as was performed during the MAA simulation. The patient will have confirmatory post-therapy imaging (PET/CT) to confirm the distribution of the radiomicrospheres 20 immediately after the therapy. Follow up of the patient is usually performed by monitoring the patient for any symptoms and by imaging approximately 3 months after the treatment.

Thus, the improved radioembolization techniques in the present invention provides an invaluable approach to extend the overall survival of patients who otherwise have few options and keep an acceptable quality of life with fewer side effects that are commonly encountered with conventional chemotherapy and current methods of RE.

Fabrication of the Radiomicrosphere

According to preferred embodiments of the present invention, fabrication of the radiomicrosphere consists of ²²⁵Ac (alpha emitter) and ⁸⁹Zr (positron emitter) both bound to a resin microsphere and verification of its stability at physiologic pH and temperature. The acceptance criteria will be a 97% or greater binding efficiency of isotopes to the resin (i.e., <3% free isotope).

In preferred embodiments of the present invention, ²²⁵Ac and ⁸⁹Zr have been chosen since they have independently been used in human subjects (in various forms, as a single isotope labeled product). The total number of particles will be designed to be 37 million (±10%,) in each dose. This number was chosen as it makes the dose more embolic than the current commercially available glass microspheres and less embolic than the current commercially available resin microspheres, thought by experts to be respective shortcomings with both products. Based on the various studies with ²²⁵Ac labeled antibodies, we will aim for the following specific activities 100 mBq ²²⁵Ac & 1 Bq ⁸⁹Zr per sphere=total 100 μCi ²²⁵Ac & 1 mCi ⁸⁹Zr.

According to preferred embodiments of the present invention, the following preparation procedure is employed for the synthesis of radiomicrospheres 20:

1) Bland cation exchange resin microspheres (similar to the Bio-rad Aminex 50W-X4, or in alternative embodiments, a resin custom synthesized with controlled degree of cross linking and select functional groups on surface) are washed with sterile water for injection and added to suitable three-neck round bottom flask with septum attached on all necks for reagent additions. Sterile water for injection (SWFI) is added and stirred using magnetic stirrer assembly to form a reasonable slurry suspension. If needed, SWFI can be added in 0.5 ml increments.

2) Simultaneously, ²²⁵Ac solution is added to the flask from one side neck and ⁸⁹Zr solution is added from other neck over few minutes while stirring is continued for next 15 minutes. (The pH of both radioactive solutions can be adjusted to desired value before addition to the flask)

3) A buffer solution is then added to the reaction vessel with further stirring (pH desired is 7±0.2).

4) The labeled radiomicrospheres 20 are then filtered and washed with SWFI. The pH of last wash solution is checked. The filtrated is then carefully collected and re-suspended in appropriately labeled vial using SWFI.

5) Activity measurement is carried out for suitable vials. A gamma counter is used to measure all the wash waste to find the ⁸⁹Zr labeling efficiency.

Stability test for ²²⁵Ac and ⁸⁹Zr will be performed as follows: radiomicrospheres 20 are re-suspended and sample of the suspension after gently agitating the suspension using push and pull of syringe plunger is extracted. The sample is diluted with 0.9 ml saline and ⁸⁹Zr gamma activity is measured as A1. The pH is retained at 7.0 (allowed range is ±0.5). The vial is then gently agitated in a water bath at 37° C. for 20 minutes. The bath is removed and the vial is centrifuged at 4 G for 2 min. A 100 μl sample of the supernatant saline solution denoted as sample B1 is taken from the vial and counted for ⁸⁹Zr in a dose calibrator or other suitable equipment such as gamma spectroscopy. The percent unbound ⁸⁹Zr can be calculated as: {Supernatant count×10/test sample count}×100=% unbound ⁸⁹Zr. If the counts fall outside the range of the measuring equipment, suitable dilutions can be performed and activity will be calculated in the light of dilution factors.

After measuring the ⁸⁹Zr gamma counts, the sample B1 is diluted by adding 0.9 ml saline and passing the solution through a Accell Plus CM cation exchange medium (300 Å, 0.35 mmol/g ligand density; Waters) with enhanced hydroxamate to trap all of ⁸⁹Zr. The column is washed with SWFI. The collected wash solution is centrifuged and a 100 μl sample from supernatant is extracted. The alpha activity is measured and the activity multiplied by 20. This value is recorded as leached ²²⁵Ac activity C1. The stock sample of A1 is gently agitated using push and pull of syringe plunger and another 100 μl sample with a syringe is extracted. The sample is passed through a suitable filter. The spheres on the filter are washed with 1 M oxalic acid to strip off all of ⁸⁹Zr and then washed with SWFI. The microspheres are then carefully isolated from the top of the filter and let dry. The ²²⁵Ac activity of these loaded microspheres is measured using alpha-particle counter with a planar silicon detector set up at predefined geometry and recorded as C2. Alternatively, measurement of the ²²⁵Ac decay product, ²¹³Bi using gamma spectrometry can be used to calculate the activity of the sample. As a second method for measurement, a setup for gamma spectroscopy unit calibrated with multiple energy counting windows of selected narrow range can be used for both ⁸⁹Zr and ²²⁵Ac activity measurement within an hour or less from a single sample. The overall leached ²²⁵Ac can be calculated as C1/C2×100=% unbound ²²⁵Ac.

The procedure may lead to inefficient radiolabeling. In case of inefficient radiolabeling, the pH of solution is adjusted to 8.5 in step 3 above. In case radiochemical purity of >97% cannot be established using the simultaneous labeling procedure as described above, a sequential labeling with standard or customized resin microspheres and modified QC testing procedure will be adopted. The entire procedure will be repeated to establish reproducibility and determine the general range of radiomicrospheres 20 loading and leaching ratios of ²²⁵Ac and ⁸⁹Zr. The simultaneous or the sequential labeling of the isotopes should lead the synthesis of stable radiomicrospheres 20. The binding efficiency of isotopes to the resin is expected to be 97% or greater (i.e., <3% isotopes freed under leaching test conditions).

Although the above description described the core concepts of the radiomicrosphere 20 in the preferred embodiments, many modifications can be made to the above described device to add additional functionality or simply perform the described method using alternative steps. As mentioned, as other isotopes are approved for human use, the isotopes may be substituted for the isotopes in the preferred embodiments. In addition, in alternative embodiments, other known fabrication techniques may be employed to manufacture the radiomicroscope 20. Still in further embodiments, the radiomicrosphere 20 may be used to treat other type of cancers besides HCC. In alternative embodiments, the radiomicrosphere 20 may be modified to have Beta emitter combined with a diagnostic isotope.

Therefore, while the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A method for the treatment of cancer cells in biological tissue using radioembolization comprising:
implanting a plurality of radiomicrospheres in a targeted treatment area, each radiomicrosphere having at least one isotope for tumoricidal therapy, wherein the at least one isotope emits primarily alpha particles.

2. The method according to claim 1, where in the at least one isotope is Actinium-225 (²²⁵Ac).

3. The method according to claim 1, further comprising a second isotope attached to the radiomicrosphere used for post-procedure dosimetry.

4. The method according to claim 3, wherein the second isotope is a positron emitter for PET dosimetry.

5. The method according to claim 4, wherein the second isotope is Zirconium-89 (⁸⁹Zr).

6. The method according to claim 3, further comprising: determining an amount of radiation absorbed dose to both tumor cells and normal liver cells using a PET scan post-procedure.

7. The method according to claim 1, wherein the plurality of radiomicrospheres in the targeted treatment area is around 37 million in each radiation treatment.

8. A method for the treatment of cancer cells in biological tissue using radioembolization comprising:
implanting a plurality of radiomicrospheres in a targeted treatment area, wherein each radiomicrosphere contains a first isotope and a second isotope, the first isotope is tumoricidal and the second isotope is for post-procedure dosimetry; and
determining a radiation absorbed dose after the radioembolization procedure is performed.

9. The method according to claim 8, wherein the first isotope is an alpha emitter for therapy.

10. The method according to claim 8, wherein the second isotope is a positron emitter for PET dosimetry.

11. The method according to claim 8, wherein the radiation absorbed dose to both tumor cells and normal liver cells after radioembolization can be determined within 5 minutes of the start of the PET scan.

12. The method according to claim 9, wherein the first isotope is Actinium-225 (²²⁵Ac).

13. The method according to claim 10, wherein the second isotope is Zirconium-89 (⁸⁹Zr).

14. The method according to claim 8, wherein the number of radiomicrospheres in the targeted treatment area is around 37 million in each radiation treatment.
